(19) United States
(12) Patent Application Publication
(10) Pub. No.: US 2010/0120060 A1
(43) Pub. Date: May 13, 2010

(54) METHOD FOR DETERMINING MOLECULES OR MOLECULE PARTS IN BIOLOGICAL SAMPLES

(76) Inventors: Lars Philipsen, Magdeburg (DE); Ansgar J. Pommer, Magdeburg (DE); Raik Böckelmann, Magdeburg (DE); Bernd Bonnekoh, Magdeburg (DE); Harald Gollnick, Magdeburg (DE)

Correspondence Address: LATIMER & MAYBERRY IP LAW, LLP, 13873 PARK CENTER ROAD, SUITE 106, HERNDON, VA, 20171, US

(21) Appl. No.: 12/282,022

(22) PCT Filed: Mar. 9, 2007
(86) PCT No.: PCT/EP2007/002087

§ 371 Date: Feb. 5, 2009

(30) Foreign Application Priority Data
Mar. 9, 2006 (DE) 10 2006 010 907.4

(51) Int. Cl. G01N 33/53

(52) U.S. Cl. 435/7.2; 977/774

(57) ABSTRACT

The invention relates to a method for determining molecules, molecule groups, and/or molecule parts in biological samples. The method includes spiking the sample at least once with at least one light emitting marker and measuring the light emission of the marker. Herein, it is intended that the light emission inherent to the sample is reduced or eliminated by means of bleaching prior to measuring the light emission of the marker.

### CROSS-REFERENCE TO RELATED APPLICATIONS

This application is a U.S. National Stage application of PCT/EP2007/002087, filed 9 Mar. 2007, the entire disclosure of which is hereby incorporated herein in its entirety by reference.

### BACKGROUND OF INVENTION

1. Field of Invention

The present invention relates to a method for determining molecules, molecule groups, and/or molecule parts in biological samples, i.e., samples of human, animal, plant, or microbial origin.

2. Description of Related Art

For a long time, the use of fluorophore-labeled antibodies for histochemical and cytochemical examination of cells has been customary. The fluorophore-labeled antibodies specifically react with antigens that are expressed on the surface of specific cells. Cells bearing such specific antigens are thus labeled with the fluorophore via the antibody. If the fluorophores are exposed to adequate excitation light, the fluorophores are induced to emit photons, which can be measured by means of a photo detector.

According to this basic binding principle, fluorescent markers, coupled to not only antibodies, but also to other detector molecules like lectins, nucleic acids, inorganic or organic molecules, probes and other ligands, are used for analyzing biological samples. Thus, the following implementations correspondingly apply not only to antibodies, but also to analogous applications using other detector molecules like lectins, nucleic acids, inorganic or organic molecules or probes and other ligands.

However, biological cell and tissue structures already genuinely contain a large number of fluorophores, whose fluorescence may have a negative influence on measurement results. In particular, this is the case if the spectrum of light that is emitted by the genuinely present fluorophores interferes with the light emission spectrum of the fluorophores of the labeling substances, for example the fluorophore-labeled antibodies.

The genuine fluorescence of cell and tissue samples is, for example, particularly disadvantageous in examining skin tissue samples. Collagen is known to be one of the endogenous skin fluorophores (Sandby-Moller, J., Thieden, E., Philipsen, P. A., Heydenreich, J., Wulf, H. C.: Skin auto-fluorescence as a biological UVR dosimeter. Photodermal Photoimmunol. Photomed. 2004, 20:33-40). Furthermore, skin components like reduced nicotinamid adenine dinucleotide phosphate, flavins, elastin, and melanin contribute to genuine skin fluorescence (König, K., Rieman, I.: High-resolution multiphoton tomography of human skin with subcellular spatial resolution and picosecond time resolution. J. Biomed. Opt. 2003, 8; 432-439). It is also a known phenomenon that, in contrast to normal skin, psoriatic plaques show red autofluorescence due to protoporphyrin IX accumulation in the horny layer (Bissonnette, R., Zeng, H., McLean, D. I., Schreiber, W. E., Roscoe, D. L., Lui, H.: Psoriatic plaque exhibit red autofluorescence that is due to protoporphyrin IX. J. Invest. Dermatol. 1998, 111:586-591).

Methods for image analysis are known in the art, by means of which removing the interfering autofluorescence from multispectral images is said to be possible. In case of strong autofluorescence of the samples or disadvantageous interference of the autofluorescence with the fluorescence of the added markers, this can lead to the loss of significant signal information, however.

Due to the genuine fluorescence of skin tissue samples it has up to date been impossible or only insufficiently possible to examine skin tissue samples in this respect in an interference-free manner using fluorophores.

### SUMMARY OF THE INVENTION

The problem underlying the solution of the present invention is to eliminate the disadvantages according to the state of the art. In particular, a method for determining molecules, molecule groups, and/or molecule parts in biological samples is provided that is particularly suitable for examining skin tissue of human or animal origin, as well as blood preparations. In accordance with the invention, a method for determining molecules, molecule groups, and/or molecule parts in biological samples is provided, comprising spiking the sample at least once with at least one light emitting marker and measuring the light emission of said marker, wherein the light emission that is inherent to the sample is reduced or eliminated by means of bleaching prior to measuring the light emission of the marker. Bleaching the sample should be conducted prior to applying the light emitting marker.

The present invention is based, at least in part, on the finding that it is possible to reduce or eliminate the light emission inherent to the sample by means of conducting a bleaching step. The bleaching step is conducted prior to the first application of a light emitting marker. Due to the reduction or elimination of the inherent light emission, the light spectrum that is emitted by the marker after having applied said marker is not influenced by light emissions coming from sources other than the markers. Here, during the course of the experiments conducted according to the present invention, it has surprisingly been found that minimizing or eliminating autofluorescence and non-specific fluorescence in cell and tissue samples including tissue sections can advantageously be achieved by means of repeated soft bleaching and is particularly useful in the analysis of skin tissue samples. Some of the experimental results supporting this can be found in the following Examples. Thus, in the method according to the present invention for minimizing the autofluorescence, the biological sample is subjected to repeated soft bleaching, i.e., including at least two bleaching steps, before the sample is spiked with a light emitting marker for the first time. Furthermore, it has been found during the conduction of the experiments that bleaching is preferably conducted by means of light having a wavelength range corresponding to or comprising the excitation wavelength of the marker(s) used, like the wavelength of 488 nm in the case of FITC. As is shown in the Examples, autofluorescence and specific fluorescence in cell and tissue samples can be achieved by means of the method according to the present invention within a relatively short time, i.e., within about two to three hours, by means of three bleaching steps using light of two wavelengths, so that the sample basically ceases to exhibit any inherent fluorescence emitting at the excitation wavelength of the light emitting marker intended for use, and is, on the other hand, intact in such a way that it can be analyzed with light emitting markers. For example, FIGS. 1 and 2 show such a result.

Herein, the term “inherent light emission” is understood to denote the emission of light by components of the sample, without light emitting substances having been added to the sample. The light emission inherent to the sample comprises the autofluorescence as well as the non-specific fluorescence of the sample, but also all further interfering light emissions, like for example, fluorescences originating from the coating material of an object carrier or from substances used for fixing the samples.

Herein, the subsequently used term “inherent fluorescence” is understood to denote the emission of light by components of the sample, without substances emitting fluorescence having been added to the sample. The fluorescence inherent to the sample comprises the autofluorescence as well as the non-specific fluorescence of the sample, but also all further interfering light emissions, like for example fluorescences originating from the coating material of an object carrier or from substances used for fixing the samples.

The term “bleaching” is understood to denote the destruction of the fluorophores contained in the sample. Destroying the fluorophores comprises, in particular, destroying the fluorophore groups of molecules of the sample, which effect inherent light emission or inherent fluorescence.

Herein, a biological sample is understood to denote samples of human, animal, plant, or microbial origin. The term “microbial”, which is derived from the term “microbe”, comprises the entire range of microorganisms, including bacteria, viruses, fungi, protozoa and metazoa, algae, cyanobacteria, and prions (cf. Pschyrembel, Klin. Wörterbuch, 259^{th} edition, de Gruyter Berlin—New York 2002, p. 1063+1064: Microbes: see microorganisms.—Microorganisms: also microbes; bacteria, viruses, protozoa, fungi (small fungi, so-called funguli, see “fungi”)). The sample can be, for example, tissue or liquids like blood, lymph, or secretions, as well as preparations made thereof. For instance, blood preparations, like cell preparations from mononuclear cells, lymphocytes, monocytes, granulocytes, thrombocytes, and erythrocytes from blood, can be examined by means of the method according to the present invention.

Thus, the invention allows for examining samples having a strong autofluorescence. The method is therefore in particular suitable for the fluorescence analysis of autofluorescent tissue samples or autofluorescent cell cultures of human or animal origin. The fluorescence analysis of autofluorescent cell cultures comprises the fluorescence analysis of both adherent cells and suspension cells. The method according to the present invention is in particular suitable for the fluorescence analysis of skin tissue of human or animal origin.

In the following, the method according to the present invention will be explained in more detail with respect to fluorescence analysis. However, this is not to be understood as limiting the method according to the present invention to the examples of fluorescence analysis given herein.

According to the present invention, the inherent fluorescence of a sample is eliminated or reduced by means of bleaching. Bleaching is conducted in such a way that the binding sites for the fluorophore-labeled markers, which are applied to the biological sample subsequently to bleaching in accordance with the present invention, are not destroyed. Bleaching the sample should be conducted prior to applying the fluorophore-labeled marker. In a preferred embodiment, bleaching the samples is conducted by means of irradiating the sample with light. The wavelength of the light is selected in such a way that the functional groups of molecules of the sample, which cause the inherent fluorescence of the sample, are destroyed.

In the case of fluorescence analysis, a light emitting marker is understood to denote a fluorophore-labeled marker. Fluorophore-labeled markers comprise, for example, fluorophore-labeled antibodies, lectins, nucleic acids, probes, and other binding molecules. The fluorophore-labeled marker may feature any type of fluorophore; the fluorophore, however, is preferably selected from the group comprising fluorescent nanoparticles (quantum dots) and fluorochromes.

In a preferred embodiment, bleaching is conducted until the signal strength of the inherent fluorescence has been reduced to a predefined level. To this end, the sample can, for example, be subjected to light of a specific wavelength for a predefined period of time. The bleaching procedure according to the present invention can be repeated several times. This is particularly advantageous in cases where the duration of light exposure that is required in order to reduce the signal strength of the inherent fluorescence to a predefined level is not known. In this case, it is preferred that several bleaching cycles are conducted, wherein each bleaching cycle comprises bleaching the sample for a predefined period of time and measuring the signal strength of the remaining inherent fluorescence. Appropriately, measuring the signal strength of the remaining inherent fluorescence is conducted after bleaching is completed.

The light used for bleaching the sample is preferably generated by means of the excitation light source of an inverted or upright fluorescence microscope. Accordingly, the light source can be, in particular, one or more mercury vapor lamps, halogen lamps, xenon lamps, lasers, light emitting diodes, or combinations thereof.

In one embodiment of the present invention, the wavelength of the light used for bleaching is selected to be similar to the excitation wavelength of a fluorophore that is supposed to be employed as a marker. Alternatively, it is also possible to select light having a wavelength range comprising the excitation wavelength of the fluorophore. By means of bleaching the biological sample with light corresponding to or comprising the wavelength of the fluorophore, the elimination of the inherent fluorescence of the sample, which is excited by said wavelength, is achieved. If the fluorophore-labeled marker that is excited at this wavelength is applied to the sample subsequently to the bleaching procedure according to the present invention, measuring the fluorescence is thus no longer disturbed by the inherent fluorescence of the sample.

In case two or more markers having different excitation wavelengths are used, the bleaching procedure according to the present invention preferably comprises bleaching with light having a wavelength range that comprises all excitation wavelengths of the employed marker. Of course, bleaching can also be conducted in n partial steps by means of exposure to light having the excitation wavelength of a first marker, subsequently by means of exposure to light having the excitation wavelength of an i^{th} marker, and finally by means of exposure to light having the excitation wavelength of the n^{th} marker.

The duration of the bleaching procedure depends on the time that is required to reduce the inherent fluorescence of the biological sample below a predefined level. Appropriately, several bleaching cycles are provided, wherein the duration of each individual bleaching step is preferably between 1 and 60 min, more preferably between 5 and 45 min, particularly preferably between 10 and 30 min.

Here, the number of bleaching cycles should be selected in such a way that the fluorescence inherent to the biological sample, which emits at a predefined excitation wavelength, is preferably at most 10%, more preferably at most 3%, most preferably at most 0.3%, in each case as related to the fluorescence intensity. This also applies if only one bleaching step is conducted.

For determining the inherent fluorescence of a sample, fluorescence measurements should first be conducted prior to performing the method according to the present invention in order to determine an indication for the required number of bleaching cycles and the required duration of a bleaching cycle.

The method is suitable for the preparation of samples that are to be analyzed by means of luminometry, microscopy, fluorescence microscopy, confocal microscopy, Multi-Epitope Ligand Cartography (MELC), flow cytometry, or Fluorescence-Activated Cell Sorting (FACS). Multi-Epitope Ligand Cartography is described in DE 197 09 348 A1 and EP 0 810 428 A1, among other places.

The term “fluorophore-labeled marker” employed herein is a synonym for the term “fluorescence-labeled marker”.

The method according to the present invention may be employed in the practical or theoretical disciplines of medicine for diagnostic purposes or for identifying novel pathogenic or therapeutic target structures or for therapy and/or drug monitoring. In the following, the invention will be explained in more detail by way of examples with reference to the drawings.

### BRIEF DESCRIPTION OF THE DRAWINGS

FIG. 1a shows a fluorescence microscopic image of skin tissue (63× magnification).

FIG. 1b shows a graphic representation of the autofluorescent alteration of defined areas of the sample shown in FIG. 1a against the number of bleaching cycles.

FIG. 2 shows a fluorescence microscopic image of lymphocytes. Panel A shows cells incubated with PBS prior to the bleaching step according to the present invention. Panel B shows the same cells subsequent to the bleaching step according to the present invention. Panel C shows fluorescence signal of the subsequent reaction with fluorophore-labeled antibody.

### DETAILED DISCUSSION OF EMBODIMENTS DEPICTED IN THE FIGURES

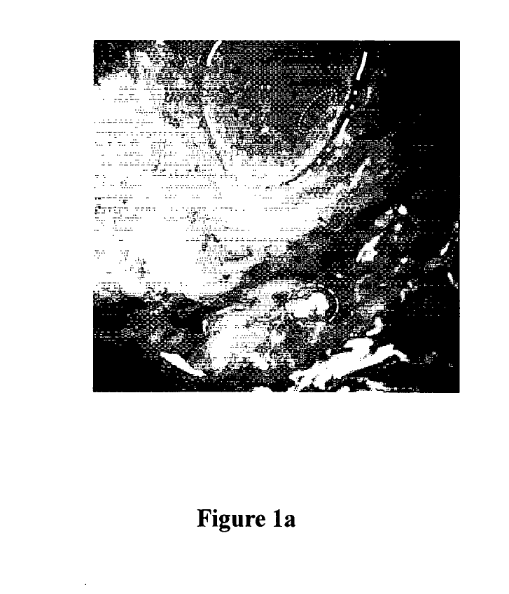

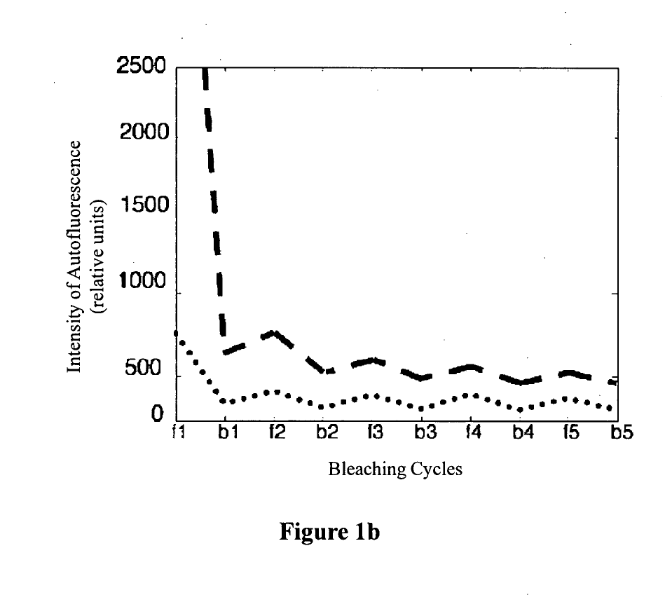

The fluorescence microscopic image of a skin tissue sample shown in FIG. 1a was obtained prior to conducting the method according to the present invention. A strong autofluorescence can be observed. For two areas of the image (circled in FIG. 1a), the fluorescence intensity is depicted against the number of bleaching cycles in FIG. 1b. Herein, the dashed curve in FIG. 1b corresponds to the area of a hair follicle of the epidermis that is defined by a dashed line in FIG. 1a, while the dotted line in FIG. 1b corresponds to the area of the dermis (corium) that is defined by a dotted line in FIG. 1a. The dash-dot-line in FIG. 1a defines the epidermis of the skin tissue sample. In FIG. 1b, the axis of ordinates shows the fluorescence intensity while the axis of abscissae the number of bleaching cycles, wherein the letters b and f used therein represent imaging after bleaching and imaging prior to bleaching, respectively. It can be seen that the signal strength of the autofluorescence is significantly reduced after five bleaching cycles.

Sample Preparation:

The sample was prepared as follows: biopsies of skin tissue from patients suffering from psoriasis as well as of healthy skin tissue were taken under local anesthesia. The biopsies had a diameter of 6 mm. After taking, the biopsies were quick-frozen. From the frozen biopsies, 5 μm sections were made by means of a cryotome. Subsequently, the sections were air-dried for 10 min at room temperature, then submerged in acetone for 10 s at room temperature, dried again, and finally stored at −20° C. Immediately prior to conducting the method according to the present invention, the sections were submerged in PBS, pH 7.4, for rehydration.

Bleaching and Fluorescence Measurements:

For conducting the bleaching cycles, the section to be examined (i.e., skin), which will be referred to as sample in the following, was applied onto an object carrier and mounted on the object stage of an inverted wide field microscope (Leica DM IRE2, with a xenon lamp for fluorescence excitation), which was equipped with fluorescence filters for fluorescein isothiocyanate (FITC) and phycoerythrin (PE). For fluorescence measurements, a CCD camera (Apogee KX4) was employed.

The sample was examined by means of fluorophore-labeled antibodies. FITC and PE were selected as fluorophores. For examining the sample, the following procedures were conducted:

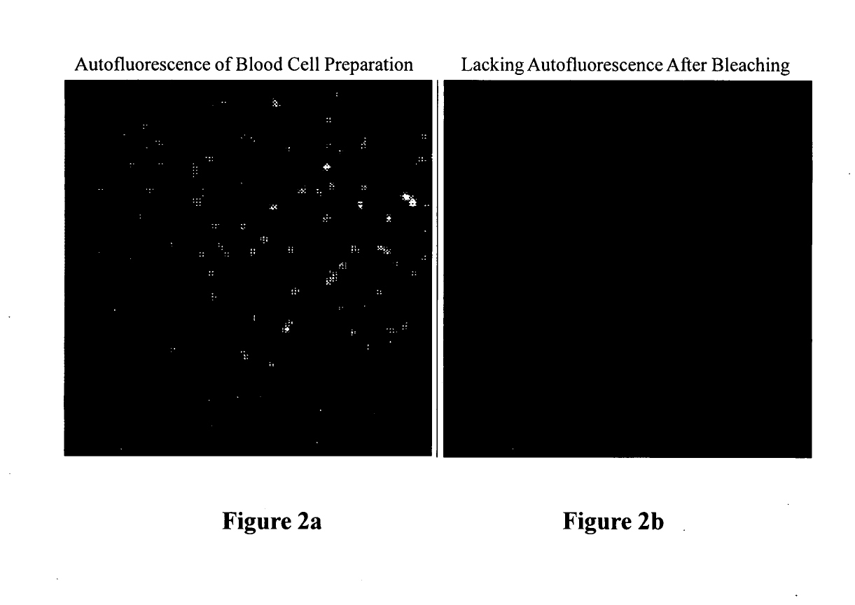

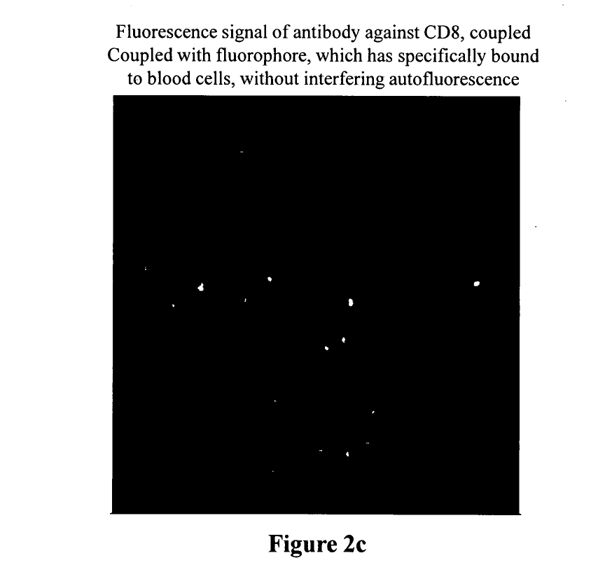

A. Eliminating the inherent fluorescence of the sample that is emitted at the excitation wavelengths of FITC and PE. Three bleaching cycles were conducted.
1.) Measuring the inherent fluorescence, wherein one image was recorded with fluorescence filter sets for each FITC and PE. Exposure time was 5 s (see FIG. 2A for FITC);
2.) a) Bleaching the sample with light having a wavelength of 488 nm (excitation wavelength of FITC) for 15 min; b) bleaching the sample with light having a wavelength of 546 nm for 15 min;
3.) Measuring the inherent fluorescence as described in step 1.);
4.) Waiting for 10 min;
5.) Measuring the inherent fluorescence as described in step 1.);
6.) a) Bleaching the sample with light having a wavelength of 488 nm (excitation wavelength of FITC) for 15 min; b) bleaching the sample with light having a wavelength of 546 nm for 15 min;
7.) Measuring the inherent fluorescence as described in step 1.);
8.) Waiting for 10 min;
9.) Measuring the inherent fluorescence;
10.) a) Bleaching the sample with light having a wavelength of 488 nm (excitation wavelength of FITC) for 15 min; b) bleaching the sample with light having a wavelength of 546 nm for 15 min;
11.) Measuring the inherent fluorescence as described in step 1.).

The result for FITC is shown in FIG. 2B. It can be seen that the sample has no inherent fluorescence that is emitted at an excitation wavelength of 488 nm.

B. Determining molecules or molecule parts of the sample using FITC-labeled antibodies and PE-labeled antibodies.

The sample obtained according to paragraph A was spiked in the manner known with a solution containing FITC-labeled antibodies, incubated, and the solution containing FITC-labeled antibodies that were not bound to the sample was removed. Subsequently, the FITC-labeled antibodies that were bound to the binding sites (antigens) on the sample were excited at 488 nm by light exposure and the emitted fluorescent radiation was measured (see FIG. 2C for FITC-labeled anti CD8 antibodies). The image obtained shows, for the first time, the light emitted by added FITC without the influence of the autofluorescence of the sample.

Subsequently to measuring, the FITC molecules that were bound via the antibodies were bleached. After bleaching, the sample can be spiked with other FITC-labeled antibodies in several cycles in the manner described above in order to determine further antigens on the sample.

Subsequently to the cycle with FITC-labeled antibodies and to bleaching the FITC molecules bound to the sample, the sample was spiked with a solution containing PE-labeled antibodies, incubated, and the solution containing PE-labeled antibodies that were not bound to the sample was removed. Afterwards, the PE-labeled antibodies that were bound to the binding sites (antigens) on the sample were excited by means of light exposure and the emitted fluorescence radiation was measured. Here, too, several cycles with different PE-labeled antibodies may be conducted.

1. A method for determining molecules, molecule groups, and/or molecule parts in a biological sample, said method comprising:
spiking the sample at least once with at least one light emitting marker; and
measuring the light emission of said marker,
wherein the light emission inherent to the sample is reduced or eliminated by means of bleaching prior to measuring the light emission of the marker, and wherein bleaching is conducted prior to spiking the sample with the at least one light emitting marker.

2. The method according to claim 1, wherein the light emission inherent to the sample comprises autofluorescence, non-specific fluorescence, or both.

3. The method according to claim 1, wherein the light emitting marker emits fluorescent light.

4. The method according to claim 1, wherein bleaching of the sample is conducted by means of exposing the sample to light.

5. The method according to claim 4, wherein the wavelength of the light is selected in such a way that the functional groups of the sample, which cause the light emission inherent to the sample, are destroyed.

6. The method according to claim 1, wherein the sample is selected from the group comprising human, animal, plant, and microbial tissue.

7. The method according to claim 1, wherein the sample is a blood sample.

8. The method according to claim 1, wherein the biological sample comprises human or animal skin tissue.

9. The method according to claim 1, wherein the biological sample is cell or tissue culture material.

10. The method according to claim 1, wherein the light emitting marker has a light emitting unit that is a fluorescent nanoparticle or fluorochrome.

11. The method according to claim 10, wherein the fluorescent nanoparticle is a quantum dot.

12. The method according to claim 1, wherein the light emitting marker comprises a light emitting unit that is conjugated to an antibody, a lectin, a probe, and/or another ligand.

13. The method according to claim 1, wherein bleaching is repeated one or more times.

14. The method according to claim 1, wherein the light emission inherent to the sample is reduced to a predefined signal strength.

15. The method according to claim 1, wherein bleaching is continued until the light emission inherent to the sample has been reduced to a predefined signal strength.

16. The method according to claim 4, wherein the light used for bleaching the sample is generated by means of one or more mercury vapor lamps, halogen lamps, xenon lamps, lasers, light emitting diodes, or combinations of these.

17. The method according to claim 1, wherein the sample is a blood preparation of human or animal origin.

18. The method according to claim 1, wherein the sample is a skin sample of human origin.

19. The method according to claim 1, wherein the sample is cell or tissue culture material of human origin.
